(19) United States
(12) Patent Application Publication
(10) Pub. No.: US 2013/0157358 A1
(43) Pub. Date: Jun. 20, 2013

(54) Method for neuroepithelial cells differentiation from pluripotent stem cells and medium using same

(71) Applicants: Hong-Lin Su, Taichung City (TW); Sheng-Mei Chen, Nantou City (TW)

(72) Inventors: Hong-Lin Su, Taichung City (TW); Sheng-Mei Chen, Nantou City (TW)

(21) Appl. No.: 13/485,914

(22) Filed: May 31, 2012

(30) Foreign Application Priority Data
Dec. 14, 2011 (TW) 100146179

(51) Int. Cl. C12N 5/0797; C12N 5/0735

(52) U.S. Cl. 435/366; 435/377

(57) ABSTRACT

The present invention discloses a neural induction medium comprising Wnt-signal agonist, TGFβ-signal inhibitor and FGF-signal agonist for inducing neural differentiation. The neural induction medium used in a culture system is capable for inducing the neural differentiation of stem cells into neuroepithelial cells which are useful for the clinical applications. Therein, the neuroepithelial cells can further differentiate into mature neurons for the practical applications including regeneration medicine and drug discovery for neural disorders.

### FIELD OF THE INVENTION

The present invention relates to a method for neuroepithelial cells differentiation and a medium using same. Specifically, the present invention discloses a method for neuroepithelial cells differentiation from pluripotent stem cells and medium using same.

### BACKGROUND OF THE INVENTION

Stem cells are the undifferentiated cells which exhibit the capacities for self-renewing and differentiating into more than two kinds of mature somatic cells. They are classified into totipotent stem cells, pluripotent stem cells, multipotent stem cell and bipotent stem cell according to their differentiation capacity. Furthermore, they cloud be also classified into embryonic stem cells, somatic stem cells and induced pluripotent stem cells (iPSCs) upon their origins. Herein, human embryonic stem cells are derived from the inner cell mass of the pre-implantation blastocyst and reveal the pluripotency for differentiating into all adult somatic cells. In addition, iPSCs are generated from the reprogrammed somatic cells by introducing the DNA plasmid or protein for the enforced expression of specific transcription factors to acquire the embryonic stem-like cells.

So far, many principle investigators have focused on the regulation and control of the stem cells differentiation due to its abilities in proliferation, self-renewing and differentiation capacities to specific tissue. They attempt to apply these stem cells in diseases therapies and regeneration medicine if these stem cells can differentiate into the destined cells, tissues or organs under regular control. For example, the dopaminergic neurons and organs acquired from culture could be applied for the treatment of Parkinson disease and organ damages, respectively.

Especially, the stem cells derived neurons are required for the biomedical investigations in studying neural development, nerve damage and neuro-degeneration diseases, and drug discovery. Therefore, how to produce the neuroepithelial cells, the precursor of neurons, with high purity in large scale is quite important for the above applications. In the previous studies, many investigators tried to induce neural differentiation of embryonic stem cells by suspension culture system with fibroblast growth factor-2 (FGF-2) at the early step of differentiation (Li, X. J. et al., 2005; Timothy et al., 2009; Xu et al., 2005; Vallier, L. et al., 2005). Although the induction culture system can acquire the neuroepithelial cells exhibiting the similar expression profile as the cells in neural tube, however, it spends more than 14 days for inducing neural differentiation. Furthermore, there are some non-neural cells contaminated with neuroepithelial cells during the adhesion process within the culture system. These contaminated non-neural cells need to be removed by enzyme digestion and manually culling out under the microscope to obtain the high purity of neuroepithelial cells.

The Smad inhibitor such as Noggin and SB431542 were chosen and added into the induction medium for shortening the time cost of the neural differentiation of stem cells (Elkabetz et al., 2008; Lee et al., 2007; Chambers et al., 2009). In addition, the genetic manipulations or co-culture with other cell lines were also performed to induce the neuronal differentiation.

Although the aforesaid methods to generate the neuroepithelial cells from stem cells, the disadvantages such as poor efficiency of neural differentiation, expansive cost, risk resulted from virus-mediated genetic manipulations and non-neural cells contamination still limit the neuroepithelial cells production in large scale. Moreover, the contaminated non-neuronal cells or undifferentiated stem cells in the cultured pool may affect the further neural differentiation. In addition to disruption on neural differentiation, the undifferentiated pluripotent stem cells bring the risk in teratoma formation in the transplanted recipients.

Therefore, efficiently induce the neural differentiation of stem cells into neuroepithelial cells, which reveal the expression of most neural markers, with high purity will benefit the further differentiation into mature neurons for improving the clinical reliability and avoiding the risk.

### SUMMARY OF THE INVENTION

In one of its aspects, the invention provides a method for neuroepithelial cells differentiation from pluripotent stem cells, comprising steps of: (a) culturing a pluripotent stem cell into an embryoid body by suspension culture; and (b) culturing the embryoid body in a first neural induction medium for generating neuroepithelial cells, wherein the first neural induction medium comprises the Wnt-signal agonist, TGFβ-signal inhibitor and FGF-signal agonist.

The method can further added with the step below after step (b):

(c) substituting the first neural induction medium to a secondary neural induction medium for promoting the further differentiation of the cultured cells into neuroepithelial cells.

Furthermore, the pluripotent stem cells may be selected from human embryo stem cells or iPSCs.

The Wnt-signal agonists may be selected from Wnt1, Wnt3a, or the pharmaceutical drug, BIO (6-bromoindirubin-3′-oxime), which inhibits the kinase activity of GSK-3β (glycogen synthase kinase-3β).

The TGF-β signal inhibitors may be selected from BMP inhibitor (bone morphogenetic protein inhibitor), Chordin, Noggin, Dorsomorphin, Smad1 inhibitor, SB431542 which is the pharmaceutical drug as Activin/Nodal receptor inhibitor, and Smad2/3-inhibitor.

The FGF-signal agonist may be selected from FGF2, ligand for FGF, ERK (extracellular signal-related kinase) activator, JNK (c-jun N-terminal kinase) activator or PI3K (phosphoinositol-3 kinase) activator.

Upon the method, it could efficiently shorten the time cost for acquiring the neuroepithelial cells from pluripotent stem cells with higher purity. The acquired neuroepithelial cells indeed exhibit the expression of neural markers and forebrain marker including Nestin, Sox1, Pax6, Zic-1, N-cadherin (neural markers) and BF-1 (forebrain marker).

In another aspect, the present invention provides a neural induction medium comprising the Wnt-signal agonist, TGFβ-signal inhibitor and FGF-signal agonist for inducing the neural differentiation of stem cells into neruoepithelial cells with high purity.

### BRIEF DESCRIPTION OF THE DRAWINGS

The patent or application file contains at least one drawing executed in color. Copies of this patent or patent application publication with color drawing(s) will be provided by the Office upon request and payment of the necessary fee

FIG. 1 shows the morphology and growth curve of the human embryonic stem cells which are cultured in TESR1 medium without feeder cells.

FIG. 2 shows the mophorlogy of the cells which are culutred by suspension culture with the first neural induction medium.

FIG. 3A shows the mophorlogy of the cultured cells under microcope observation with 100× magnification.

FIG. 3B shows the mophorlogy of the cultured cells under microcope observation with 200× magnification.

FIG. 3C shows the mophorlogy of the cultured cells under microcope observation with 400× magnification.

FIG. 4A shows the mophorlogy of the differentiated neuroepithelial cells which adhere on the culture dish and aggregate to form the neural tube-like structure.

FIG. 4B shows the mophology of the enlogating cells which are subjected from FIG. 4A and cultured for two days.

FIG. 5A shows the expression pattern of Oct4, Nestin, Sox2, Nanog and Zo-1 in the cultured neuroepithelial cells are examined by immunofluorescence staining with the corresponding primary antibodies.

FIG. 5B shows the expression pattern of Sox1, Pax6, Zic1 and N-cadherin in the cultured neuroepithelial cells are examined by immunofluorescence staining with the corresponding primary antibodies.

FIG. 5C shows the expression pattern of BF1 in the cultured neuroepithelial cells is examined by immunofluorescence staining with the corresponding primary antibody.

FIG. 5D shows the expression pattern of Tuj1 on the axon of the adherent neuroepithelial cells is examined by immunofluorescence staining with the corresponding primary antibody.

### DETAILED DESCRIPTION OF THE INVENTION

The present invention provides a method for neuroepithelial cells differentiation from pluripotent stem cells, comprising steps of: (a) culturing a pluripotent stem cell into an embryoid body by suspension culture; and (b) culturing the embryoid body in a first neural induction medium for generating neuroepithelial cells, wherein the first neural induction medium comprises the Wnt-signal agonist, TGFβ-signal inhibitor and FGF-signal agonist.

The present invention also provides the method which further includes step (c) below after step (b) of substituting the first neural induction medium to a secondary neural induction medium for promoting the further differentiation of the cultured cells into neuroepithelial cells.

The present invention further provides a medium as the neural induction medium using in the method, comprising Wnt-signal agonist, TGFβ-signal inhibitor and FGF-signal agonist to induce the neural differentiation of pluripotent stem cells into neuroepithelial cells.

According to one embodiment of the present invention, the method for neuroepithelial cells differentiation from pluripotent stem cells and medium using same can induce neural differentiation of pluripotent stem cells into neuroepithelial cells (FIG. 2).

According to another embodiment of the present invention, more than 90% of undifferentiated pluripotent stem cells would successfully differentiate to neuroepithelial cells.

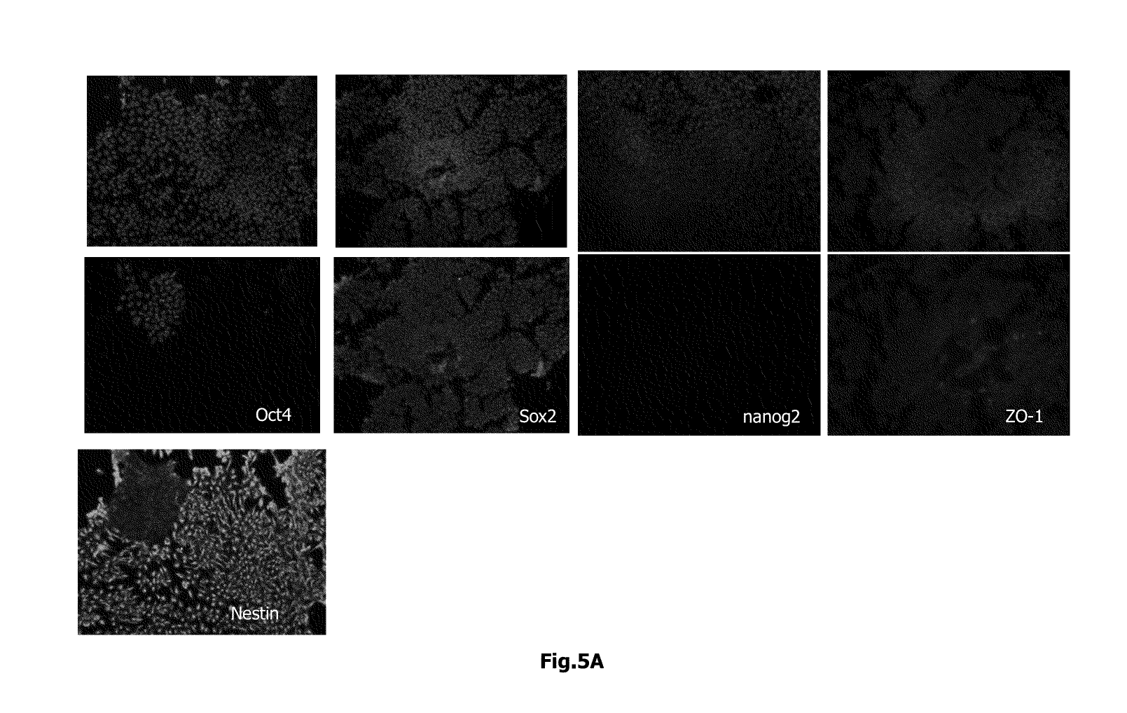

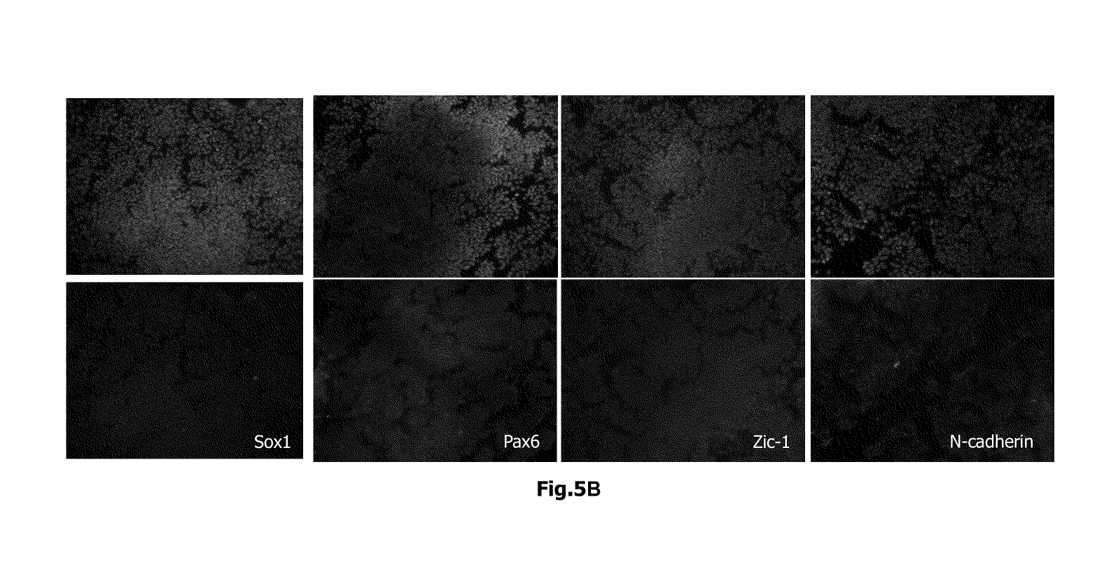

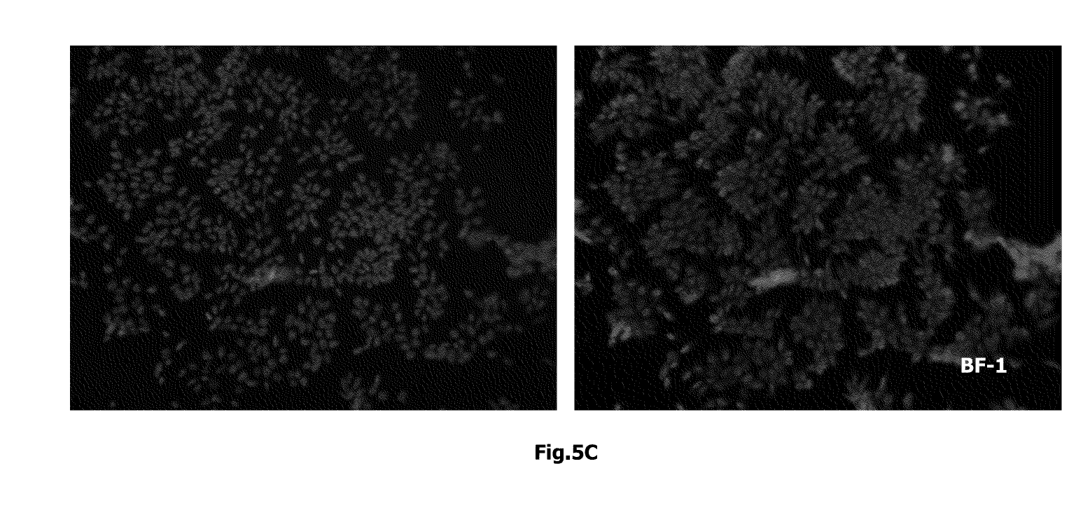

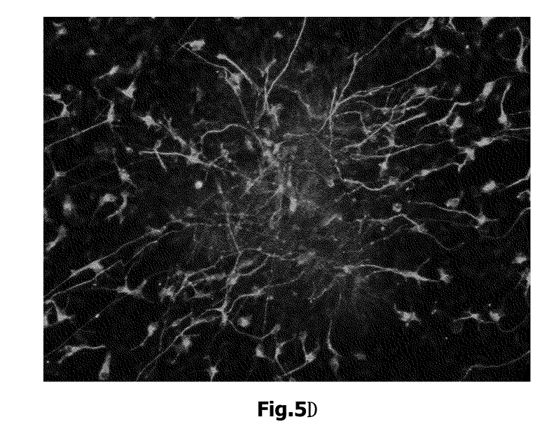

According to an example of the present invention, the neuroepithelial cells highly express neural markers including Nestin, Sox1, Pax6, Zic-1 and N-cadherin, and forebrain marker such as BF-1 (forebrain marker) within one week (FIG. 5A˜D).

Definition of the terms as used herein as below:

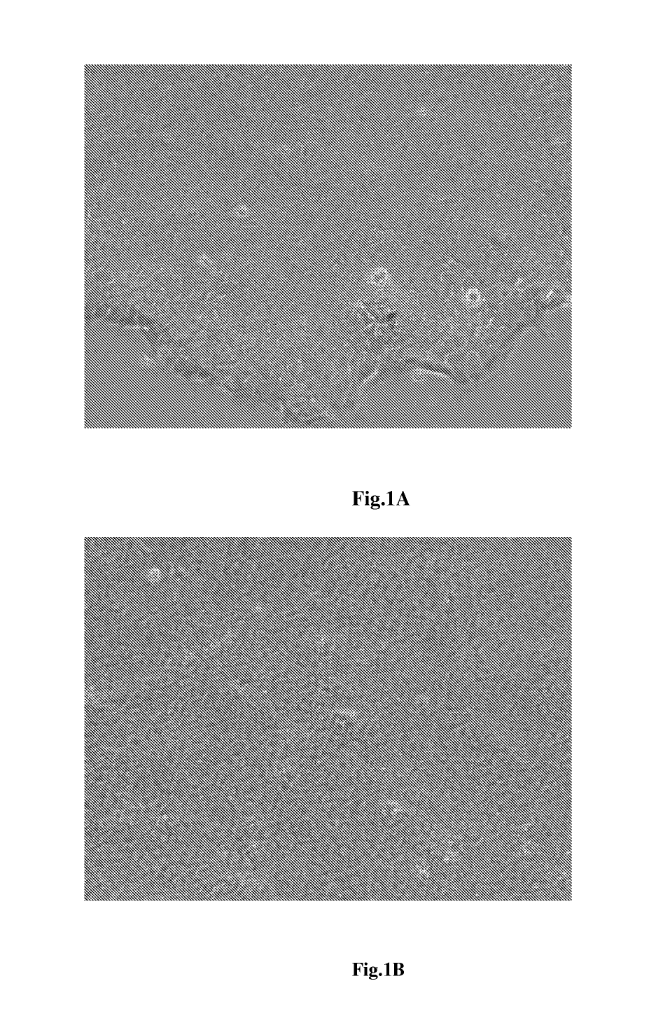

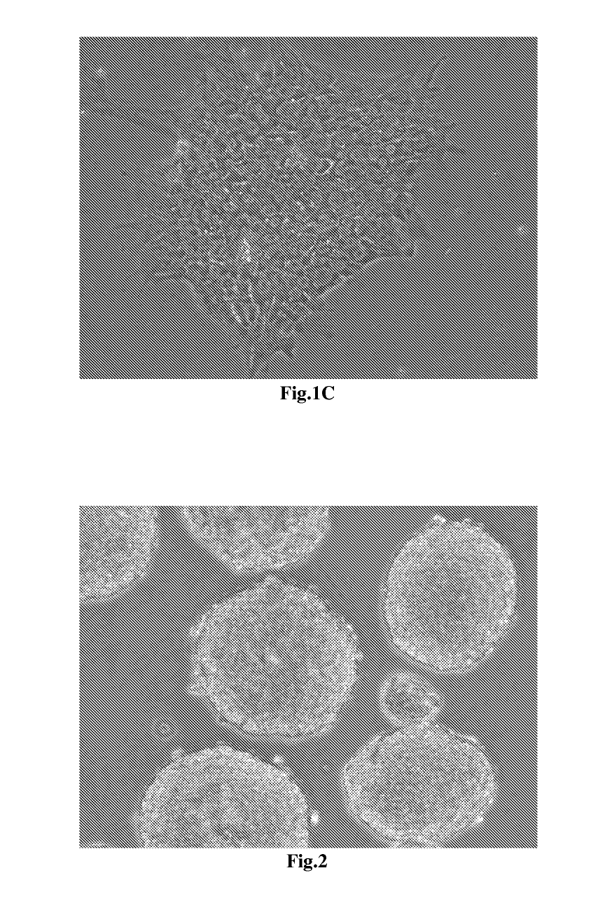

The term “pluripotent stem cells” refers to include the mammalian embryonic stem cells, iPSCs prepared by introducing exogenous expression of transgene or protein, and other pluripotent stem cells owing the differentiation potency to all kinds of somatic cells. The pluripotent stem cells, TW1 cells, used in the following examples of the present invention is a kind of human embryonic stem cells. FIG. 1A to 1C reveal the growth curve of the TW1 cells cultured in mTESR1 medium without feeder cells.

The term “neural markers” refers to the genes such as Nestin, Sox1, Pax6, Zic-1 and N-cadherin are expressed in the neuroepithelial cells, hence, the expression of these neural markers are good indicators to verify whether the pluripotent stem cells have differentiated into neuroepithelial cells.

The term “forebrain markers” is a transcription factor expressed in the neurons of forebrain, such as BF1 (brain factor 1). Therefore, the expression of this forebrain marker is used to verify whether the pluripotent stem cells have differentiated into neuroepithelial cells.

The term “embryonic stem cells markers” refers to the transcription factors such as Oct4 and Nanog are highly expressed in the embryonic stem cells. Therefore, the expressions of these embryonic stem cell markers are applied to examine the efficiency of neural differentiation of pluripotent stem cells into nueropithelial cells.

The term “neuroepithelial cells” presents as globular appearance and organize with the neighbor cells by tightly tubular attachment around the edge to form the petal structure called as neural rosettes.

The term “Wnt-signal agonist” is a kind of the GSK-3β inhibitors that are capable for stabilizing β-catenin through inhibiting the kinase activity of GSK-3β. The Wnt-signal agonists include Wnt1, Wnt3a, or GSK-3β inhibitor BIO, wherein the Wnt-signal agonist used in the following examples of the present invention is GSK-3β inhibitor BIO which exhibits the chemical formula as C₁₆H₁₀BrN₃O₂ and the structure as

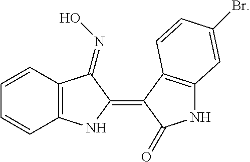 [SMILES: O=C1Nc2cc(Br)ccc2/C1=C1/Nc2ccccc2/C1=N\O]

The term “TGFβ-signal inhibitor” is capable to reduce the self-renewing ability and Oct4 expression of stem cells. The TFGβ antagonists include bone morphogenetic protein inhibitor, Chordin, Noggin, Dorsomorphin, Smad1 inhibitor, Activin/Nodal receptor inhibitor or Smad2/3-inhibitor. Herein, the TGFβ antagonist used in the following examples of the present invention is TGFβ-signal suppressing drug, SB431542, which is the Activin/Nodal receptor inhibitor exhibiting the chemical formula as C₂₂H₁₆N₄O₃ and molecular structure as

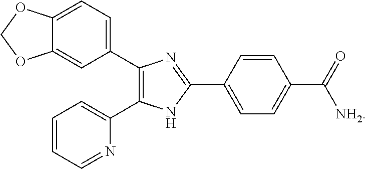 [SMILES: NC(=O)c1ccc(-c2nc(-c3ccc4c(c3)OCO4)c(-c3ccccn3)[nH]2)cc1]

The term “FGF-signal agonist” includes FGF2, ligand of FGF receptor, ERK (extracellular signal-related kinase) activator, JNK (c-jun N-terminal kinase kinase) activator or PI3K (phosphoinosital-3 kinase) activator. Herein, the FGF-signal agonist used in the following example of the present invention is FGF2 that is capable for activating the Ras/Erk signaling pathway.

The following non-limiting examples are provided to further illustrate the present invention.

### Example 1

### Generation of Embryoid Body from Embryonic Stem Cells

The human embryonic stem cells, TW1 cells, are cultured at 37° C. and 5% CO₂, first. Following the pre-culture, the aggregated clones of embryonic stem cells are selected for the further suspension culture within DMEM-F12 containing 20% knock-out replacement serum (KSR, Invitrogen, USA) at 37° C. and 5% CO₂ for two days to generate the embryoid bodies.

### Example 2

### Induction of the Neural Differentiation of Pluripotent Stem Cells into Neuroepithelial Cells

The embryoid bodies generated in example 1 are collected in the 15 mL centrifuge tube and placed at room temperature for descending the embryoid bodies and discarding the supernatant. Prepare 500 mL of first neural induction medium which contains the basic constitutions listed in table 1 and additive drugs including 0.5 μM BIO, 10 μM SB431542 and 10 ng/ml FGF2.

Notably, the announcement has to be emphasized here is that the working concentration of these additive drugs in the first neural induction medium are not restricted on what we indicated. The working concentration of BIO is between 0.05 μM to 0.5 μM; the working concentration of SB431542 is between 1 μM to 100 μM; and the working concentration of FGF2 is between 1 ng/ml to 100 ng/ml. The collected cells are further cultured with the first neural induction medium for two days to generate neuroepithelial cells as shown in FIG. 2. In FIG. 2, the globular neuroepithelial cells reveal the tightly tubular arrangement at the edge.

TABLE 1
The constitution of primary induction medium:

| Constitutes | Content |
|---|---|
| DMEM medium (GIBCO, Lot no. 11965-092) | 326 ml |
| DMEM-F12 (GIBCO, Lot no. 11765-054) | 163 ml |
| N2 Supplement (GIBCO, Lot no. 17502-048) | 5 ml |
| MEM non-essential amino acid (Lot. M7145, Sigma) | 5 ml |
| Heparin (1 mg/ml) | 1 ml |

### Example 3

### Further Induction of Neural Differentiation into Neuroepithelial Cells

The neuroepithelial cells gained in example 2 are switched from first neural induction medium to secondary neural induction medium which is added with 10 ng/ml FGF2 for maintaining the further differentiation into neuroepithelial cells. Herein, the constitutions of secondary neural induction medium are shown in table 2.

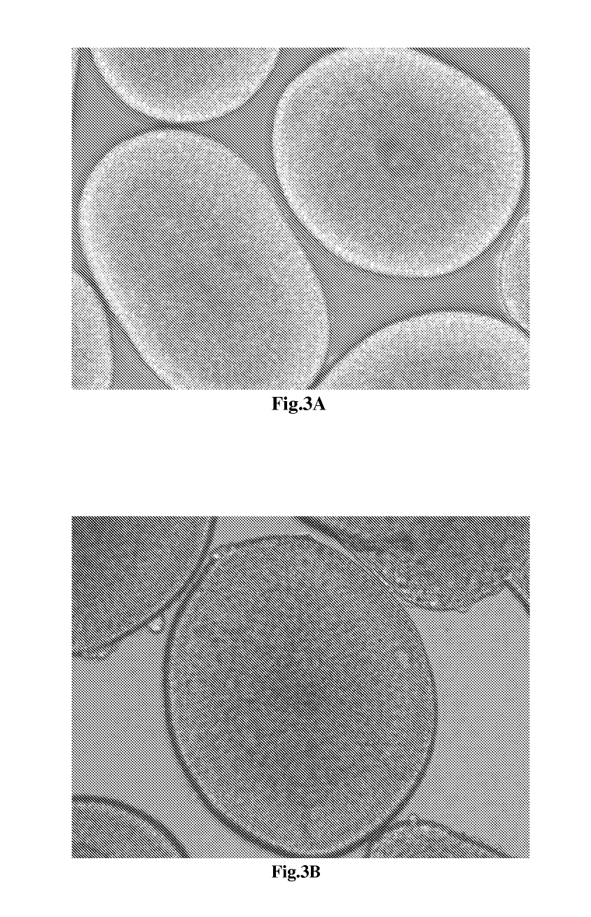

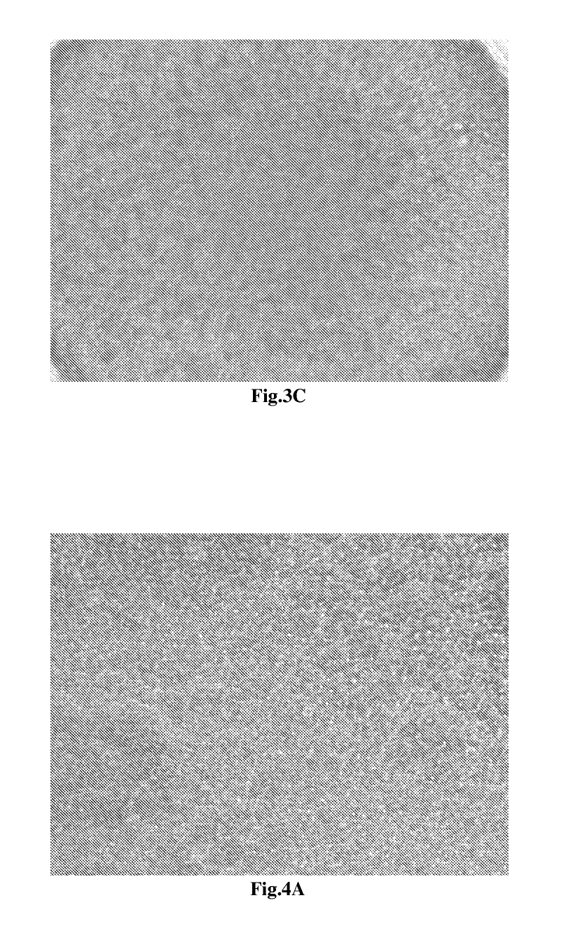

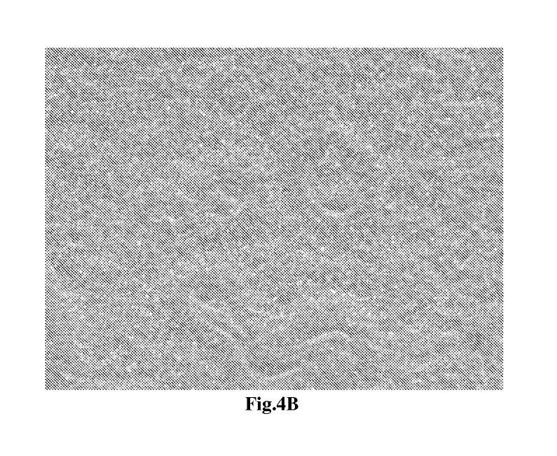

The cells finishing the neural differentiation are shown in FIG. 3 and FIG. 4 with 100×, 200×, and 400× magnifications under microscope. In FIG. 3A, the cultured cells reveal the homogenous morphology and form the globular structure units through tightly tubular aggregation at the edge. The FIG. 3B reveals the globular structure unit which contain the tightly tubular aggregation at the edge and rosette formations resembling the early neural tube in the center. The FIG. 3C shows that the cultured cells in the center of the globular unit form rosette clump resembling the neural tube-like structure. The FIG. 4A shows the morphology of attaching neuroepithelial cells within the neural tube-like rosette clump; the FIG. 4B reveals the cell morphology which are cultured from FIG. 4A for two days and exhibit the elongation. Therefore, according to the examination of the morphology of the cells cultured from example 1 to example 3, these cells acquired from this induction culture are neuroepithelial cells.

TABLE 2
The secondary neural induction medium

| Constitutions | Content |
|---|---|
| Neurobasal medium (GIBCO,) | 500 ml |
| N2-supplement (GIBCO,) | 5 ml |
| Non-essential amino acid (NEAC, Sigma) | 5 ml |
| Heparin (1 mg/ml) | 1 ml |

### Example 4

### Preparation of Neuroepithelial Cells for the Immunofluorescence Staining

The 4-wells plate containing the cover slides is loaded with Matrigel for 6 hours to coat the cover slides which is followed by removing Matrigel and PBS wash. And then, the neuroepithelial cells acquired in example 3 are seeded on coating cover slides and cultured at 37° C., 5% CO₂ for one day to make the neuroepithelial cells adhere on the slides and exhibit the morphology as rose-ring neural tube structure.

### Example 5

### Preparation of Primary Antibodies and Secondary Antibodies

In order to identify whether the cultured cells are neuroepithelial cells, we performed the immunofluorescence staining with appropriated primary and corresponding secondary antibodies for characterization. Therefore, the cultured cells are incubated with primary antibodies diluted in PBS, which contains 3% horse serum, at 4° C. for 24 hours. Following the incubation with primary antibodies, the cells are further incubated with corresponding fluorescence conjugated secondary antibodies with 1:500 dilution in dark at room temperature for one hour. Herein, the antibodies with suitable dilution ratio and the corresponding secondary antibodies are listed in table 3.

TABLE 3
Antibodies used in the immunofluorescence staining

| | Species origin | | |
|---|---|---|---|
| Primary | of primary | | Secondary |
| antibody | antibody | Dilution | antibody |
| Oct4 | Goat | 1:200 | Cy3, FITC |
| Sox2 | Rabbit | 500 | Cy3, FITC |
| Nanog | Rabbit | 500 | Cy3, FITC |
| Nestin | Rabbit | 1:200 | Cy3, FITC |
| Pax6 | Rabbit | 1:200 | Cy3, FITC |
| Sox1 | Goat | 1:200 | Cy3, FITC |
| Zic-1 | Rabbit | 1:200 | Cy3, FITC |
| N-cadherin | Mouse | 1:200 | Cy3, FITC |
| ZO-1 | Rabbit | 1:100 | Cy3, FITC |
| BF-1 | Rabbit | 1:100 | Cy3, FITC |
| Tuj1 | Mouse | 1:500 | Cy3, FITC |

### Example 6

### Identification of Neuroepithelial Cells by Immunofluorescence Staining

After discarding medium and PBS wash, the cells seeded on the cover slides according to the method of example 4 were fixed with 4% paraformaldehyde. For cell fixation, the cells were fixed with 200 μl of 4% paraformaldehyde at 4° C. for 5 minutes that is followed by and PBS wash. Permeating cell membrane of fixed cells by 200 μl of PBS buffer containing 0.3% Triton at 4° C. for 5 minutes, and followed by PBS washing for 5 minutes thrice. Blocking the non-specific staining by incubation with 5% horse serum diluted in PBS at room temperature for one hour. Removing the blocking reagent and adding the primary antibodies prepared in example 5 for staining of the neural markers and forebrain marker in the cultured cells. After primary antibodies staining and PBS wash, the corresponding fluorescence conjugated secondary antibodies are added for recognizing the primary antibodies and are followed by PBS wash. Adding 200 μl PBS buffer containing 1 μg/ml DAPI to staining the cellular nuclei in dark at room temperature for 10 minutes, which is followed by PBS wash and mounting. The protein expression of various neural markers and forebrain marker were observed under fluorescent microscope and calculated by the software, AlphaEaseFC.

The protein expression observed by the fluorescent microscope is showed in FIG. 5, herein, the FIG. 5A reveals the expression of Oct4, Nestin, Sox2, Nanog and Zo-1 on the tenth day of induction culture according to the fluorescence signal under microscope. Moreover, FIG. 5B shows the expression of Sox1, Pax6, Zic1 and N-cadherin on the tenth day of the induction culture according to the fluorescence signal; FIG. 5C reveals the expression of BF1, the forebrain marker, in the cells on the tenth day of induction culture according fluorescence signal; and FIG. 5D reveals the expression of Tuj1, which is expressed in the neuronal axon, on the adherent neuroepithelial cells according to the fluorescence signal. According to the soft ware analysis, the table 4 shows the highly expression of the neural markers and forebrain marker in the culture cells on the tenth day of induction culture. In contrast, the expression of Nanog, the marker stands for stem cells, is absent in the cultured cells.

TABLE 4
The expression of stem cells, neural and forebrain markers

| Oct4 | nanog | Sox2 | Nestin | Zo-1 | N-cadherin | Sox1 | Pax6 | Zic-1 | BF1 |
|---|---|---|---|---|---|---|---|---|---|
| 6.7% | 0% | 98% | 93.3% | 98% | 98% | 87.9% | %67 | 89% | 95.6% |

Hence, the cells cultured from example 1 to example 3 actually express the neural markers and forebrain marker that are associated with down-regulation of the stem cell marker and expression of neuronal axon protein according to FIG. 5A to 5D. Therefore, the induction method described from example 1 to example 3 in this invention is able to generate the neuroepithelial cells with high purity.

The inventors have clearly demonstrated that the method and the medium of the present invention can not only shortens the induction time cost, but also increases the purity of the neural differentiation of pluripotent stem cells into neuroepithelial cells which highly express neural markers. Following, the neuroepithelial cells could be further induce to differentiate into mature neurons for the application in regeneration medicine and drug discovery to neural disorders.

Without departing from the spirit and scope of the present invention, in view of the present disclosure, anyone skilled in the art may make various changes and modification to the components of medium or the steps of method, which falls the protected scope of the present invention.

Notably, it is understood that the present invention is not limited to the particular embodiments disclosed, but it is intended to cover modifications within the spirit and scope of the present invention as defined by the claims.

We claims:

1. A neural induction medium comprises Wnt-signal agonist, TGFβ-signal inhibitor and FGF-signal agonist.

2. The neural induction medium of claim 1, wherein the Wnt-signal agonist is selected from the group consisting of Wnt ligands and GFK-3β inhibitor (glycogen synthase kinase 3β inhibitor) BIO (6-bromoindirubin-3′-oxime).

3. The neural induction medium of claim 1, wherein the Wnt-signal agonist is GFK-3β inhibitor BIO with the working concentration between 0.05 μM to 50 μM.

4. The neural induction medium of claim 2, wherein the Wnt-signal agonist is GFK-3β inhibitor BIO with the working concentration between 0.05 μM to 50 μM.

5. The neural induction medium of claim 1, wherein the TGFβ-signal inhibitor is selected from the group consisting of bone morphogenetic protein inhibitor (BMP inhibitor), Chordin, Noggin, Dorsomorphin, Smad1 inhibitor, Activin/Nodal receptor inhibitor SB431542, and Smad2/3-inhibitor.

6. The neural induction medium of claim 1, wherein the TGFβ-signal inhibitor is Activin/Nodal receptor inhibitor SB431542 with working concentration between 1 μM to 100 μM.

7. The neural induction medium of claim 5, wherein the TGFβ-signal inhibitor is Activin/Nodal receptor inhibitor SB431542 with working concentration between 1 μM to 100 μM.

8. The neural induction medium of claim 1, wherein the FGF-signal agonist is selected from the group consisting of FGF2, FGFR ligand, ERK (extracellular signal-related kinase) activator, JNK (c-jun N-terminal kinase kinase) activator and PI3K (phosphoinosital-3 kinase) activator.

9. The neural induction medium of claim 1, wherein the FGF-signal agonist is FGF2 with working concentration between 1 ng/ml to 10 ng/ml.

10. The neural induction medium of claim 8, wherein the FGF-signal agonist is FGF2 with working concentration between 1 ng/ml to 10 ng/ml.

11. A use of the neural induction medium of claim 1 in inducing the neural differentiation of pluripotent stem cells into neuroepithelial cells.

12. A method for neuroepithelial cells differentiation from pluripotent stem cells comprising steps of:
(a) culturing a pluripotent stem cell into an embryoid body by suspension culture;
(b) culturing the embryoid body in a first neural induction medium for generating neuroepithelial cells, wherein the first neural induction medium is selected from the group consisting of claim 1 to claim 10.

13. The method for neuroepithelial cells differentiation from pluripotent stem cells of claim 12, further including a step (c) below after step (b):
(c) substituting the first neural induction medium to a secondary neural induction medium for promoting the further differentiation.

14. The method for neuroepithelial cells differentiation from pluripotent stem cells of claim 12, wherein the pluripotent stem cell of step (a) is selected from a group consisting of human embryonic stem cell and iPSC (induced pluripotent stem cell).

15. The method for neuroepithelial cells differentiation from pluripotent stem cells of claim 13, wherein the pluripotent stem cell of step (a) is selected from a group consisting of human embryonic stem cell and iPSC (induced pluripotent stem cell).
